(19) United States
(12) Patent Application Publication
(10) Pub. No.: US 2026/0232086 A1
(43) Pub. Date: Aug. 13, 2026

(54) HOOK-AND-LOOP FASTENER

(71) Applicant: YKK Corporation, Tokyo (JP)

(72) Inventors: Wataru Kuriyama, Toyama (JP); Isamu Michihata, Toyama (JP); Ayumi Fujisaki, Toyama (JP); Yui Miyawaki, Toyama (JP)

(21) Appl. No.: 19/531,835

(22) Filed: Feb. 6, 2026

(30) Foreign Application Priority Data
Feb. 12, 2025 (JP) 2025-020770

(51) Int. Cl. A44B 18/00; A61F 5/02

(52) U.S. Cl. CPC A44B 18/0015; A44B 18/0011; A44B 18/0073; A61F 5/028

(57) ABSTRACT

A hook-and-loop fastener includes: a base; and engaging elements and projections provided on a surface on one side of the base. Each engaging element includes: a stem projecting from the surface of the base; and an engaging head projecting from the stem in a first direction that is one direction along the surface of the base. The engaging elements are arrayed in the first direction to form an element array(s). The element arrays are arranged on the surface of the base with a space between adjacent ones of the element arrays in a second direction orthogonal to the first direction. Each of the projections are arranged in any of spaces between adjacent ones of the element arrays. The arranged projections form a visible portion that allows a user to visually recognize any one direction along the surface of the base.

### CROSS REFERENCE TO RELATED APPLICATIONS

The entire disclosure of Japanese Patent Application No. 2025-020770 filed Feb. 12, 2025 is expressly incorporated by reference herein.

### TECHNICAL FIELD

The present invention relates to a hook-and-loop fastener.

### BACKGROUND ART

Hook-and-loop fasteners that can be removably attached to a loop member having a plurality of loops have been known. Among hook-and-loop fasteners, there are types that have directionality in engagement with the loop member (see, for example, Literature 1: International Publication No. WO 2018/150493).

The hook-and-loop fastener disclosed in Literature 1 includes a sheet-shaped base and a plurality of engaging elements provided on an upper surface of the base. Each engaging element includes a stem projecting from the upper surface of the base, and an engaging head projecting from an upper end of the stem in one direction along the upper surface of the base. In this hook-and-loop fastener, directionality in engagement with the loop member arises because the engaging heads of the plurality of engaging elements have the same projecting direction. Specifically, when a force is applied to the hook-and-loop fastener in the projecting direction of the engaging heads, the engaging elements are likely to catch on the loops, whereas when a force is applied in the direction opposite to the projecting direction of the engaging heads, the engaging elements are likely to disengage from the loops.

When attaching a hook-and-loop fastener of the type described above to any product, it is desirable to correctly orient the hook-and-loop fastener after checking the direction in which the engaging heads of the hook-and-loop fastener project. However, because the engaging heads of the hook-and-loop fastener are small, it is difficult to visually discern the projecting direction of the engaging heads with the naked eye, and the task of checking the attachment orientation of the hook-and-loop fastener is labor-intensive and time-consuming.

### SUMMARY OF THE INVENTION

An object of the invention is to provide a hook-and-loop fastener that simplifies checking its attachment orientation.
[1] A hook-and-loop fastener according to an aspect of the invention, including: a sheet-shaped base; and a plurality of engaging elements and a plurality of projections provided on a surface on one side of the base, each of the engaging elements including: a stem projecting from the surface of the base; and an engaging head projecting from the stem in a first direction that is one direction along the surface of the base, the engaging elements being arrayed in the first direction to form an element array, the element array including a plurality of element arrays, the element arrays being arranged on the surface of the base with a space between adjacent ones of the element arrays in a second direction orthogonal to the first direction, the space including a plurality of spaces on the surface of the base, each of the projections being arranged in any of the spaces between adjacent ones of the element arrays, the arranged projections forming a visible portion that allows a user to visually recognize any one direction along the surface of the base.
[2] In [1] above, the visible portion is preferably a pattern having directionality.
[3] In [1] or [2] above, the visible portion preferably points in the first direction or a direction opposite to the first direction.
[4] In any of [1] to [3] above, a projection height of the projections from the surface of the base is preferably lower than a projection height of the engaging elements from the surface of the base.
[5] In any of [1] to [4] above, it is preferable that each of the projections is arranged between the stems of the engaging elements adjacent in the second direction, and an arrangement range of each of the projections in the first direction is narrower than an arrangement range of each of the engaging elements in the first direction.
[6] In any of [1] to [5] above, a dimension of each of the projections in the second direction is preferably larger than a dimension of each of the engaging elements in the second direction.
[7] Preferably, the hook-and-loop fastener according to any of [1] to [6] above further includes a reinforcement rib arranged on at least one side of each of the engaging elements in the second direction, in which a dimension of each of the projections in the first direction is larger than a dimension of the reinforcement rib in the first direction.
[8] In any of [1] to [7] above, it is preferable that the base has a rectangular shape with a longitudinal direction of the base in the first direction, the visible portion includes a plurality of visible portions, and the visible portions are arranged on the base at a predetermined interval in the first direction.

### BRIEF DESCRIPTION OF DRAWINGS

FIG. 1 is a plan view of a hook-and-loop fastener according to an exemplary embodiment of the invention.

FIG. 2 is a partial perspective view of the hook-and-loop fastener according to the exemplary embodiment.

FIG. 3 is a partial sectional view taken along line III-III in FIG. 1.

FIG. 4 is a partial sectional view taken along line IV-IV in FIG. 1.

FIG. 5 schematically illustrates a support using the hook-and-loop fastener according to the exemplary embodiment.

FIG. 6 is a partial perspective view of a hook-and-loop fastener according to a modified example.

FIG. 7 is a partial sectional view of the hook-and-loop fastener according to the modified example.

### DETAILED DESCRIPTION

Referring to the drawings, an exemplary embodiment of the invention will be described.

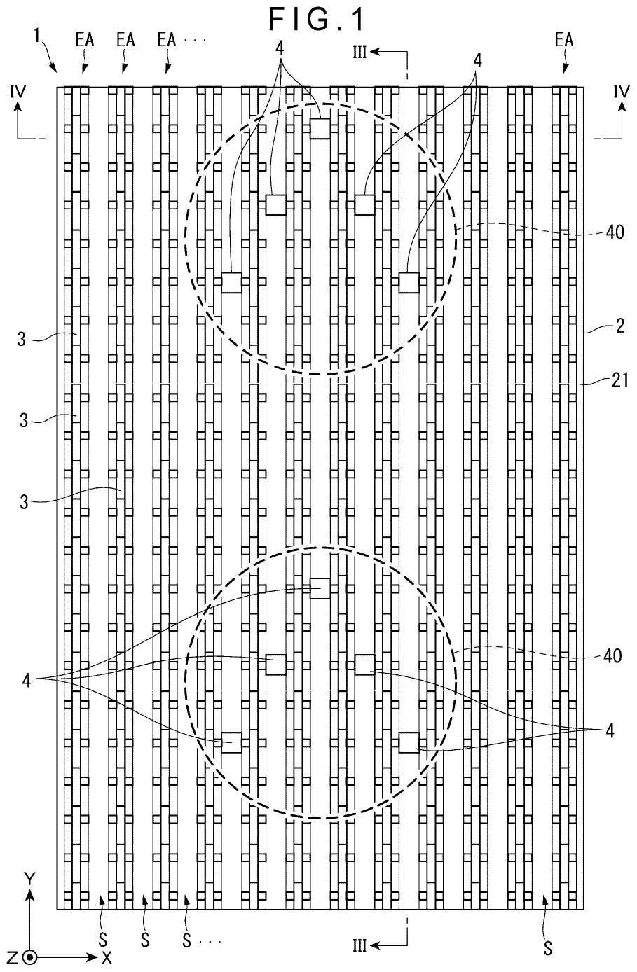

As illustrated in FIG. 1, a hook-and-loop fastener 1 of the exemplary embodiment has a rectangular sheet shape integrally formed by resin molding. The hook-and-loop fastener 1 can be removably attached to a loop member (not illustrated) having a plurality of loops. Although not particularly limited, the hook-and-loop fastener 1 is suitably attached, together with the loop member, to a product removably put on a user's body, such as a support.

Dimensions of the hook-and-loop fastener 1, such as a length, width, and thickness, are not particularly limited, and the size and shape of the hook-and-loop fastener 1 can be changed as appropriate by cutting or the like. Types of synthetic resins forming the hook-and-loop fastener 1 are not particularly limited, and it is possible to use, for example, thermoplastic resins such as polypropylene, polyester, nylon, polybutylene terephthalate, or copolymers thereof.

For convenience of explanation, X, Y, and Z directions, which are orthogonal to each other, will be used below. In the exemplary embodiment, the X direction is a width direction of the hook-and-loop fastener 1, the Y direction is a length direction of the hook-and-loop fastener 1, and the Z direction is a thickness direction of the hook-and-loop fastener 1. The Y direction is the same as a machine direction along which the hook-and-loop fastener 1 is conveyed during production.

### Configuration of Hook-and-Loop Fastener

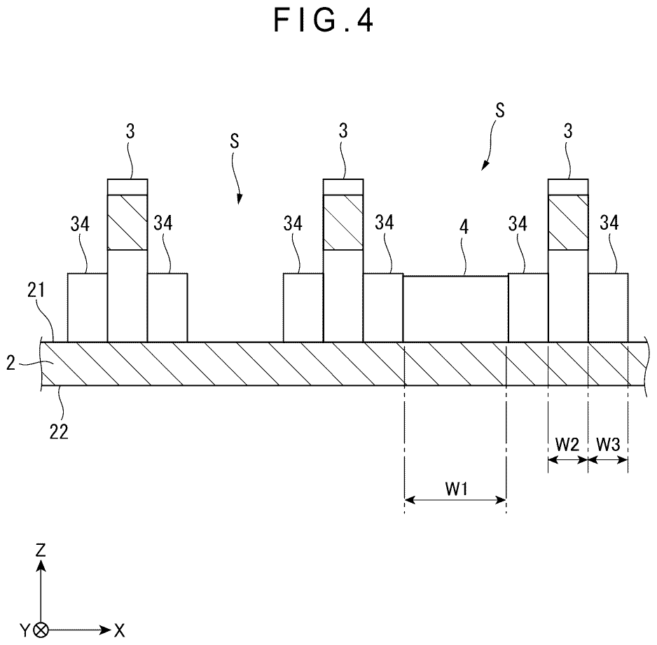

As illustrated in FIGS. 1 to 4, the hook-and-loop fastener 1 of the exemplary embodiment includes a base 2, and a plurality of engaging elements 3 and a plurality of projections 4 provided on the base 2.

The base 2 has a sheet shape having the same length and width as those of the hook-and-loop fastener 1. The base 2 has a thickness that provides moderate flexibility and strength. The base 2 has a pair of surfaces 21 and 22 facing opposite sides in the Z direction. The pair of surfaces 21 and 22 are flat and parallel to each other. In the exemplary embodiment, the surface 21 faces a first side (+Z side) in the Z direction and the surface 22 faces a second side (−Z side) in the Z direction.

The engaging elements 3 as a whole project from the surface 21 of the base 2. The engaging elements 3 each have a hook shape curved so that its projecting end is oriented toward a first side (+Y side) in the Y direction. Specifically, each engaging element 3 includes a stem 31 projecting from the surface 21 of the base 2 and an engaging head 32 projecting from an end of the stem 31 toward the +Y side. The engaging element 3 has a predetermined width in the X direction (X-directional dimension W2).

The stem 31 has a front side surface 311 facing the +Y side and a rear side surface 312 facing a second side (−Y side) in the Y direction. The rear side surface 312 is inclined in the Z direction such that a portion of the stem 31 that is located farther from the base 2 is positioned closer to the engaging head 32.

The engaging head 32 curves to slightly extend toward the −Z side while extending from the end of the stem 31 toward the +Y side. The engaging head 32 and the front side surface 311 of the stem 31 form a concave portion 321 for housing the loop of the loop member. Further, the engaging head 32 and the rear side surface 312 of the stem 31 of another engaging element 3 that is adjacent on the +Y side form a loop path RP for guiding the loop of the loop member to the concave portion 321.

When the hook-and-loop fastener 1 engages with the loop member, each loop of the loop member passes the loop path RP between the engaging elements and is housed in the concave portion 321 of the engaging element 3 located on the −Y side of that loop path RP. As a result, the engaging element 3 comes into engagement with the loop.

The plurality of engaging elements 3 are arranged in an array along both the X and Y directions. A space S is defined between the engaging elements 3 adjacent in the X direction. In other words, the plurality of engaging elements 3 form an element array EA by being arrayed in the Y direction. A plurality of element arrays EA are arranged in the X direction with the space S therebetween. The engaging elements 3 are arranged such that the stems 31 are opposed to each other in the X direction with the space S interposed therebetween.

The hook-and-loop fastener 1 according to the exemplary embodiment further includes a pair of reinforcement ribs 34 provided for each engaging element 3. The pair of reinforcement ribs 34 are provided on opposite sides in the X direction of the stem 31 of the engaging element 3, which reinforces the stem 31. Each reinforcement rib 34 projects from the surface 21 of the base 2 and is formed integrally with the stem 31. The reinforcement rib 34 has a roughly rectangular parallelepiped shape and has predetermined dimensions in the X, Y, and Z directions.

In the exemplary embodiment, an X-directional dimension W3 of the reinforcement rib 34 is not particularly limited, and is substantially equal to the X-directional dimension W2 of the engaging element 3. Further, a Y-directional dimension D3 of the reinforcement rib 34 is smaller than a minimum width in the Y direction of the stem 31. Furthermore, a projection height H3 of the reinforcement rib 34 from the surface 21 of the base 2 (i.e., a maximum dimension in the Z direction of the reinforcement rib 34) is smaller than a projection height H2 of the engaging element 3 from the surface 21 of the base 2 (i.e., a maximum dimension in the Z direction of the engaging element 3).

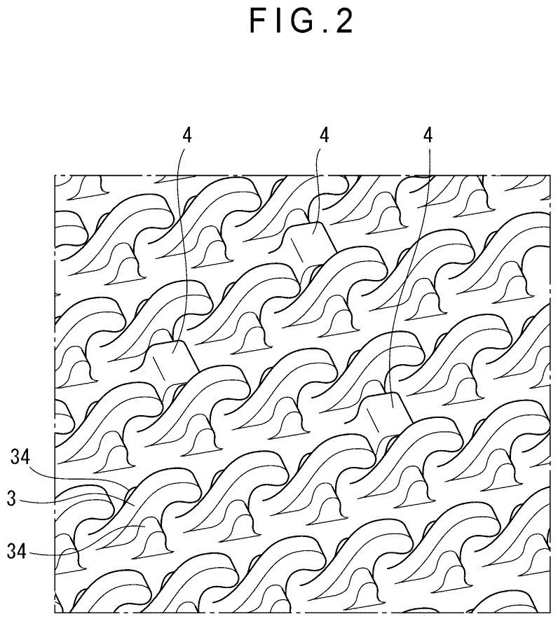

The projections 4 are arranged and configured to be visible in the hook-and-loop fastener 1. An exemplary arrangement and configuration of the projections 4 will be described with reference to FIGS. 2 to 4.

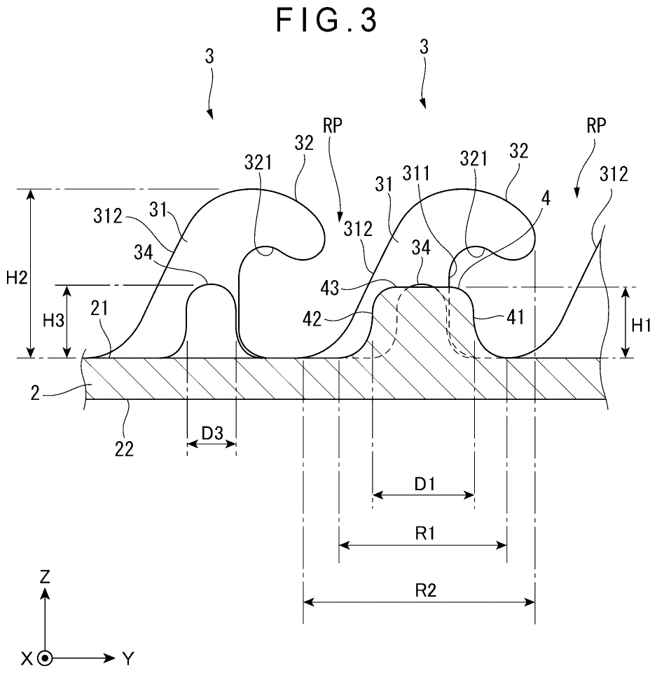

Each of the projections 4 is arranged in any of spaces S between adjacent ones of the element arrays EA. Specifically, each of the projections 4 is arranged between the stems 31 of any engaging elements 3 adjacent in the X direction. Here, an arrangement range R1 of the projection 4 in the Y direction is within an arrangement range R2 of the engaging element 3 in the Y direction. In particular, the projection 4 is preferably arranged on the −Y side with respect to the projecting end of the engaging head 32. Further, when the hook-and-loop fastener 1 is viewed in the X direction (see FIG. 3), the projection 4 preferably overlaps neither with the rear side surface 312 of the stem 31 of the engaging element 3 nor with the concave portion 321 of the engaging element 3.

The projection 4 has a roughly rectangular parallelepiped shape and has predetermined dimensions in the X, Y, and Z directions. The projection 4 has a pair of side surfaces 41 and 42 facing opposite sides in the Y direction and an upper surface 43 facing the +Z side. An R-shaped interior angle is defined between the surface 21 of the base 2 and each of the side surfaces 41, 42, and an R-shaped exterior angle is defined between the upper surface 43 and each of the side surfaces 41, 42.

An X-directional dimension W1 of the projection 4 is equal to a dimension of the space S between the engaging elements 3 adjacent in the X direction. Therefore, the projection 4 may be formed integrally with the reinforcement ribs 34 adjacent in the X direction. Further, it is preferable that the X-directional dimension W1 of the projection 4 is larger than the X-directional dimension W2 of the engaging element 3, and that the X-directional dimension W1 is at least twice the X-directional dimension W2.

For example, when the X-directional dimension W2 of the engaging element 3 according to the exemplary embodiment is 0.3 mm, the X-directional dimension W3 of the reinforcement rib 34 is 0.3 mm and the X-directional dimension W1 of the projection 4 is 0.8 mm.

In the exemplary embodiment, a projection height H1 of the projection 4 from the surface 21 of the base 2 is set such that the projection 4 is visible in the hook-and-loop fastener 1 and interference of the projection 4 with the engagement of the engaging element 3 can be prevented. Specifically, in order to prevent the projection 4 from being buried between the engaging elements 3 and becoming invisible, the projection height H1 of the projection 4 is preferably at least ⅓ of the projection height H2 of the engaging element 3. Further, in order to prevent the projection 4 from coming into contact with the loop member before the engaging element 3, the projection height H1 of the projection 4 is preferably equal to or less than the projection height H2 of the engaging element 3. For example, when the projection height H2 of the engaging element 3 is 1.2 mm, the projection height H1 of the projection 4 is preferably in a range of 0.4 mm to 1.2 mm, more preferably in a range of 0.8 mm to 1.1 mm.

In the exemplary embodiment, a Y-directional dimension D1 of the projection 4 (i.e., a dimension between the pair of side surfaces 41, 42 of the projection 4) is set to achieve a suitable balance between the visibility of the projection 4 and the flexibility of the hook-and-loop fastener 1. In particular, the Y-directional dimension D1 of the projection 4 is preferably in a range of 0.2 mm to 1.0 mm, more preferably in a range of 0.4 mm to 0.8 mm. Setting the Y-directional dimension D1 of the projection 4 at 0.2 mm or more achieves good visibility with the naked eye, and setting the Y-directional dimension D1 of the projection 4 at 1.0 mm or less achieves sufficient flexibility of the hook-and-loop fastener 1.

In the exemplary embodiment, in order to more suitably prevent the interference of the projection 4 with the engagement of the engaging element 3, the projection height H1 and the Y-directional dimension D1 of the projection 4 are adjusted in the above dimension ranges. Specifically, the Y-directional dimension D1 of the projection 4 is set larger than the projection height H1 of the projection 4. For example, when the projection height H2 of the engaging element 3 is 1.2 mm, the projection height H1 of the projection 4 is 0.5 mm, and the Y-directional dimension D1 of the projection 4 is 0.7 mm.

In the exemplary embodiment, the Y-directional dimension D1 of the projection 4 is preferably larger than the Y-directional dimension D3 of the reinforcement rib 34. Further, the projection height H1 of the projection 4 may be smaller than the projection height H3 of the reinforcement rib 34 from the surface 21 of the base 2, and the Z-directional position of the upper surface 43 of the projection 4 is preferably positioned on the −Z side with respect to the projecting end of the engaging head 32.

The projections 4 described above are arranged so that a predetermined number of projections 4 are in a predetermined pattern, as illustrated in FIG. 1. The arrangement of the predetermined number of projections 4 forms one visible portion 40. With the arrangement of the predetermined number of projections 4, the visible portion 40 has a regularity that allows a user to visually recognize any one direction along the surface 21 of the base 2.

The visible portion 40 of the exemplary embodiment is a pattern or design having directionality. The visible portion 40 of the exemplary embodiment forms an arrowhead shape by arranging the predetermined number of projections 4. The visible portion 40 points in the +Y direction. That is, the visible portion 40 points in the same direction as the projecting direction of the engaging heads 32.

Referring to FIG. 1, the arrowhead shape, which is the design of the visible portion 40 of the exemplary embodiment, will be explained in more detail. Assuming that the arrangement in the X direction of the projections 4 in the visible portion 40 is defined as a row, the visible portion 40 includes three rows of projections 4 arranged in the X direction and spaced in the Y direction from the +Y side, from the first row to the third row. An interval equivalent to one engaging element 3 is provided between adjacent ones of the rows. In the visible portion 40, the first row includes one projection 4 in the space S at a center portion of the visible portion 40 in the X direction. The second row includes two projections 4 in spaces S on opposite sides of the center portion of the visible portion 40 in the X direction. An interval equivalent to two element arrays EA and one space S is provided between the two projections 4. The third row includes two projections 4 on opposite sides of the center portion of the visible portion 40 in the X direction. An interval equivalent to four element arrays EA and three spaces S (i.e., an interval larger than the interval between the projections 4 in the second row) is provided between the two projections 4. The total of the five projections 4 thus achieves the visible pattern, which is the arrowhead shape pointing in the +Y direction.

The visible portion 40 may further include a fourth and subsequent rows of the projections 4 arranged in the X direction. The visible portion 40 may have a larger arrowhead shape by gradually increasing the interval between the two projections 4 in each row of the fourth and subsequent rows. Alternatively, the third and subsequent rows of the projections 4 may be omitted in the visible portion 40. An interval equivalent to a plurality of engaging elements 3 may be provided between adjacent ones of the rows.

It is only required that the hook-and-loop fastener 1 includes at least one visible portion 40. However, as illustrated in FIG. 1, the hook-and-loop fastener 1 may include a plurality of visible portions 40. For example, when the base 2 has a tape shape with its longitudinal direction in the Y direction, the base 2 may include a plurality of visible portions 40 at predetermined intervals in the Y direction. The arrangement of the visible portions 40 may be determined according to a cutting cycle of the hook-and-loop fastener 1.

### Example of Use of Hook-and-Loop Fastener

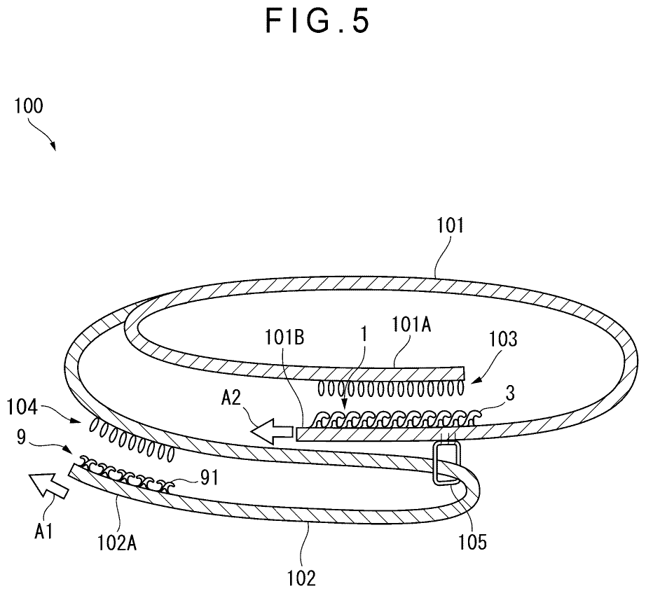

Referring to FIG. 5, an example of use of the hook-and-loop fastener 1 according to the exemplary embodiment will be described. FIG. 5 schematically illustrates a support 100 (e.g., a back brace), which is an example of use of the hook-and-loop fastener 1 according to the exemplary embodiment. It is assumed that the base 2 of the hook-and-loop fastener 1 has a rectangular shape with its longitudinal direction in the Y direction.

The support 100 includes: a first band 101 that is wrapped around a user's body; a second band 102 extending from the middle of the first band 101; a first loop member 103 attached to a first end 101A of the first band 101; the hook-and-loop fastener 1 attached to a second end 101B of the first band 101; a second loop member 104 attached to the middle of the second band 102; a standard hook-and-loop fastener 9 attached to an end 102A of the second band 102; and a ring member 105 attached near the second end 101B of the first band 101.

In the support 100, the hook-and-loop fastener 1 is arranged to be removably attached to the first loop member 103. The hook-and-loop fastener 1 is arranged so that the engaging heads 32 project toward a first side in a longitudinal direction of the first band 101 (direction from the second end 101B of the first band 101 toward the first end 101A).

In the support 100, the standard hook-and-loop fastener 9 is arranged to be removably attached to the second loop member 104. It is only required that the standard hook-and-loop fastener 9 be a hook-and-loop fastener having a typical configuration. For example, the standard hook-and-loop fastener 9 includes a plurality of engaging elements 91 each of which has a pair of engaging heads projecting toward opposite sides in a longitudinal direction of the standard hook-and-loop fastener 9.

When a user wears the support 100, the user wraps the first band 101 around the user's body, and engages the hook-and-loop fastener 1 with the first loop member 103. This temporarily fixes the support 100 to the user's body. At this time, a force is applied, due to a repulsive force from the user's body, to the hook-and-loop fastener 1 in a direction in which the engaging heads 32 are caught in loops of the first loop member 103, thereby strongly engaging the hook-and-loop fastener 1 with the first loop member 103. The support 100 is, therefore, not likely to come off the user's body.

Next, the second band 102 is passed through the ring member 105 and then folded back. The end 102A of the second band 102 is then pulled (see an arrow A1 in FIG. 5) to tighten the first band 101 around the user's body. At this time, a force is applied to the hook-and-loop fastener 1 via the ring member 105 toward a second side in the longitudinal direction of the first band 101 (direction from the first end 101A of the first band 101 toward the second end 101B), and the hook-and-loop fastener 1 moves in that direction (see an arrow A2 in FIG. 5). This movement direction of the hook-and-loop fastener 1 is a direction in which the engaging heads 32 disengage from the loops, and the hook-and-loop fastener 1 easily moves relative to the first loop member 103.

After that, the user engages the standard hook-and-loop fastener 9 with the second loop member 104. Accordingly, the support 100 is fixed to the user's body. During use of the support 100, similar to the temporary fixing described above, the hook-and-loop fastener 1 is strongly engaged with the first loop member 103 and the support 100 is not likely to come off the user's body.

As described above, when the hook-and-loop fastener 1 of the exemplary embodiment is used in the support 100, the directionality relating to the engagement of the hook-and-loop fastener 1 with the first loop member 103 provides functions of preventing the support 100 from coming off the user's body and facilitating the tightening adjustment to suit the user's body. In order to produce the support 100 that properly exhibits these functions, it is important to correctly orient the hook-and-loop fastener 1 when the hook-and-loop fastener 1 is attached to the first band 101 of the support 100.

### Effects and Advantages of the Exemplary Embodiment

As described above, the hook-and-loop fastener 1 of the exemplary embodiment includes the sheet-shaped base 2, the plurality of engaging elements 3 and the plurality of projections 4 provided on the surface 21 on one side of the base 2. Each of the engaging elements 3 includes the stem 31 projecting from the surface 21 of the base 2; and the engaging head 32 projecting from the stem 31 in a first direction (+Y direction) that is one direction along the surface 21 of the base 2. The engaging elements 3 arrayed in the first direction form the element array EA. The element arrays EA are arranged on the surface 21 of the base 2 with the space S between adjacent ones of the element arrays EA in a second direction (X direction) orthogonal to the first direction, the space S including a plurality of spaces on the surface 21 of the base 2. Each of the projections 4 is arranged in any of the spaces S between adjacent ones of the element arrays EA. The arrangement of the projections 4 forms the visible portion 40 that allows a user to visually recognize any one direction along the surface 21 of the base 2.

With this configuration, the visible portion 40 is provided by the arrangement of the projections 4 that can be visually recognized in the hook-and-loop fastener 1. Associating the direction visually recognized by the user based on the visible portion 40 in advance with the projecting direction of the engaging heads 32 allows the user to visually recognize the visible portion 40 and determine the projecting direction of the engaging heads 32. Therefore, when the hook-and-loop fastener 1 is attached to a product, a worker can easily check the attachment orientation of the hook-and-loop fastener 1.

The visible portion 40 of the exemplary embodiment is a pattern having directionality. Specifically, the visible portion 40 of the exemplary embodiment points in the first direction (+Y direction).

With such a configuration, the worker can more intuitively confirm the attachment orientation of the hook-and-loop fastener 1.

In the exemplary embodiment, the projection height H1 of the projection 4 from the surface 21 of the base 2 is lower than the projection height H2 of the engaging element 3 from the surface 21 of the base 2.

With such a configuration, it is possible to prevent the interference of the projections 4 with the loops engaged with the engaging elements 3, and to prevent a decrease in engagement force of the hook-and-loop fastener 1.

Each of the projections 4 of the exemplary embodiment is arranged between the stems 31 of the engaging elements 3 adjacent in the second direction (X direction). The arrangement range R1 of the projection 4 is narrower than the arrangement range R2 of the engaging element 3 in the first direction (+Y direction).

With such a configuration, the projections 4 are prevented from overlapping in the X direction with the loop path RP between the engaging elements 3 adjacent in the Y direction. It is therefore possible to suitably prevent the interference of the projections 4 with the loops engaged with the engaging elements 3, and to prevent a decrease in engagement force of the hook-and-loop fastener 1.

In the exemplary embodiment, the dimension in the second direction (X-directional dimension W1) of the projection 4 is larger than the dimension in the second direction (X-directional dimension W2) of the engaging element 3.

Such a configuration gives the projections 4 in the hook-and-loop fastener 1 a distinctive appearance, thus improving the visibility of the visible portion 40.

The hook-and-loop fastener 1 of the exemplary embodiment further includes the reinforcement ribs 34 arranged on at least one side of the engaging elements 3 (+X side and/or −X side in the exemplary embodiment) in the second direction. The dimension in the first direction (Y-directional dimension D1) of the projection 4 is larger than the dimension in the first direction (Y-directional dimension D3) of the reinforcement rib 34.

Such a configuration gives the projections 4 in the hook-and-loop fastener 1 a distinctive appearance, thus improving the visibility of the visible portion 40.

The base 2 of the exemplary embodiment has a rectangular shape with its longitudinal direction in the first direction (+Y direction). The visible portions 40 are arranged in the base 2 in the first direction (+Y direction) at predetermined intervals.

With such a configuration, after the hook-and-loop fastener 1 is produced, it is possible to cut the hook-and-loop fastener 1 for the intended use so that the visible portions 40 are arranged on the cut pieces.

### Modifications

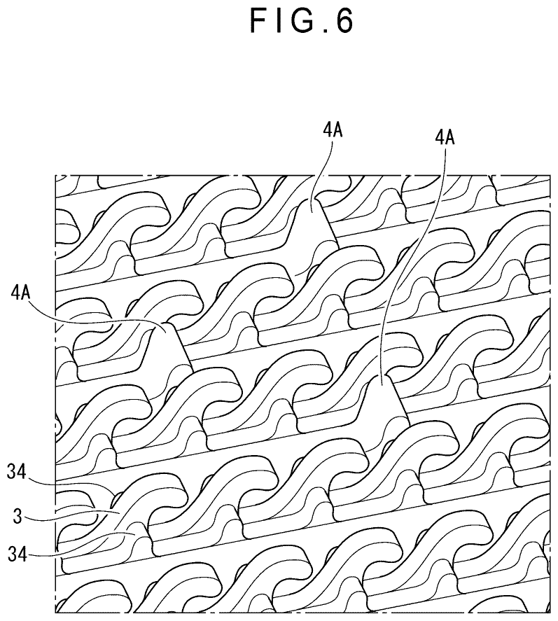

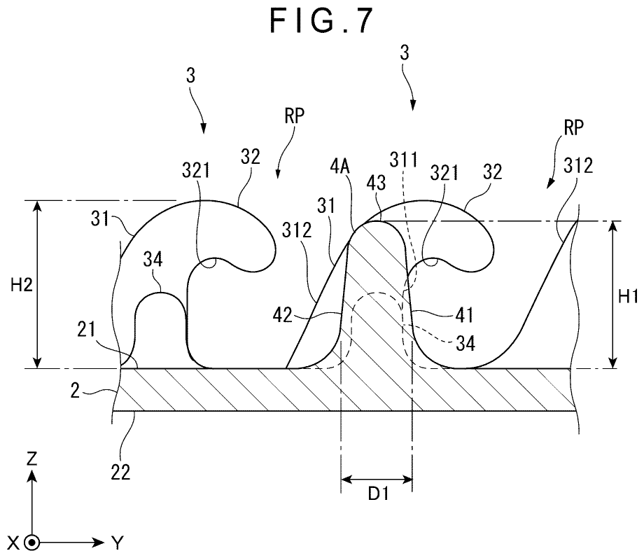

FIGS. 6 and 7 illustrate projections 4A according to a modified example. In this modified example, the projection height H1 of the projection 4A is set larger than the Y-directional dimension D1 of the projection 4A. Here, the projection height H1 of the projection 4A is preferably larger than the projection height H3 of the reinforcement rib 34, and also preferably at least ⅔ of the projection height H2 of the engaging element 3. For example, when the projection height H2 of the engaging element 3 is 1.2 mm, the Y-directional dimension D1 of the projection 4A is 0.4 mm, and the projection height H1 of the projection 4A is 1.05 mm.

In the modified example, the preferred dimensional ranges can be the same as those described in the above exemplary embodiment. In the modified example, it is possible to suitably prevent the interference of the projections 4A with the engagement of the engaging elements 3 similarly to the above exemplary embodiment. Further, the projection height H1 of the projection 4A is large in the modified example, thus providing higher visibility than that of the above exemplary embodiment.

In addition, the projections 4 of the above exemplary embodiment are not limited to the dimensions, arrangement, and configuration described above, and may be varied as appropriate. For example, if the engagement force required for the hook-and-loop fastener 1 is sufficiently achieved, the projections 4, 4A may be arranged to interfere with the loops engaged with the engaging elements 3.

The projections 4 of the above exemplary embodiment are not limited to have a roughly rectangular parallelepiped shape, and may have any other shape. For example, the projections 4 may have a similar shape to the engaging elements 3. In other words, the engaging elements 3 positioned out of the element arrays EA that serve as the projections 4, may form the visible portion 40.

In the exemplary embodiment, the pointing direction indicated by the visible portion 40 is the same as the projecting direction of the engaging heads 32. The invention, however, is not limited thereto. For example, if a worker attaching the hook-and-loop fastener 1 to a product understands a relationship between the pointing direction indicated by the visible portion 40 and the projecting direction of the engaging heads 32, the pointing direction indicated by the visible portion 40 may be different from the projecting direction of the engaging heads 32. For example, the pointing direction indicated by the visible portion 40 may be a direction opposite to the projecting direction of the engaging heads 32 or a direction forming a predetermined angle with respect to the projecting direction of the engaging heads 32.

In the above exemplary embodiment, the visible portion 40 is a pattern of an arrowhead shape. The invention, however, is not limited thereto. For example, the visible portion 40 may have any directional design, such as a heart or crescent moon. The projections 4 forming the visible portion 40 may be located on the outline of the pattern, or on the outline of the pattern and inside the pattern. Alternatively, the visible portion 40 is not limited to the pattern, but may be an array of the projections 4 having a regularity that allows the user to visually recognize any one direction.

In the exemplary embodiment, the example in which the hook-and-loop fastener 1 is used in the support 100 has been described. The hook-and-loop fastener of the invention, however, can be used in any other products.

What is claimed is

1. A hook-and-loop fastener, comprising:
a sheet-shaped base; and
a plurality of engaging elements and a plurality of projections provided on a surface on one side of the base,
each of the engaging elements comprising:
a stem projecting from the surface of the base; and
an engaging head projecting from the stem in a first direction that is one direction along the surface of the base,
the engaging elements being arrayed in the first direction to form an element array,
the element array comprising a plurality of element arrays, the element arrays being arranged on the surface of the base with a space between adjacent ones of the element arrays in a second direction orthogonal to the first direction, the space comprising a plurality of spaces on the surface of the base, each of the projections being arranged in any of the spaces between adjacent ones of the element arrays, and
the arranged projections forming a visible portion that allows a user to visually recognize any one direction along the surface of the base.

2. The hook-and-loop fastener according to claim 1, wherein the visible portion is a pattern having directionality.

3. The hook-and-loop fastener according to claim 1, wherein the visible portion points in the first direction or a direction opposite to the first direction.

4. The hook-and-loop fastener according to claim 1, wherein a projection height of the projections from the surface of the base is lower than a projection height of the engaging elements from the surface of the base.

5. The hook-and-loop fastener according to claim 1, wherein each of the projections is arranged between the stems of the engaging elements adjacent in the second direction, and
an arrangement range of each of the projections in the first direction is narrower than an arrangement range of each of the engaging elements in the first direction.

6. The hook-and-loop fastener according to claim 1, wherein a dimension of each of the projections in the second direction is larger than a dimension of each of the engaging elements in the second direction.

7. The hook-and-loop fastener according to claim 1, further comprising a reinforcement rib arranged on at least one side of each of the engaging elements in the second direction, wherein
a dimension of each of the projections in the first direction is larger than a dimension of the reinforcement rib in the first direction.

8. The hook-and-loop fastener according to claim 1, wherein the base has a rectangular shape with a longitudinal direction of the base in the first direction,
the visible portion includes a plurality of visible portions, and
the visible portions are arranged on the base at a predetermined interval in the first direction.
